(19) United States
(12) Patent Application Publication
(10) Pub. No.: US 2006/0155216 A1
(43) Pub. Date: Jul. 13, 2006

(54) Disposable urinary aid

(76) Inventor: Werner Flueckiger, Oberkulm (DE)

Correspondence Address: DAVID TOREN, ESQ.;ABELMAN FRAYNE & SCHWAB, 666 THIRD AVENUE, NEW YORK, NY, 10017-5621, US

(21) Appl. No.: 10/524,342

(22) PCT Filed: Aug. 13, 2002
(86) PCT No.: PCT/CH02/00441

§ 371 Date: Feb. 11, 2005

(51) Int. Cl. A61B 5/00

(52) U.S. Cl. 600/584; 600/573

(57) ABSTRACT

The disposable aid is formed as a folded drain member (21) with walls of a hygiene paper which is formed by folding along a central line. The drain member (21) contains an inlet section (22) for the reception of urine, a drain section (23) for the draining of urine into a toilet or the like and a sideways projecting holding part (24) which is connected to the inlet section (22). The inlet section (22) is formed of two wall sections of the same design (26; 27) and can be formed through it into a funnel. A plurality of drain members (21) can be manufactured individually or in roll form and packed into a dispenser. The drain members can be made at favourable cost and enable a comfortable, hygienically advantageous use as well as a simple disposal.

The invention relates to a disposable aid for urination in accordance with the preamble of claim 1.

The invention starts from an aid which is known from EP 1 078 614. The aid is conceived as a drain member and is formed by two walls which lie flat against each other, of an absorbent hygiene paper which can soften and/or decompose in water, and which bound a drain channel. The drain member includes an inlet section with a marginal portion which can be placed against the body of the respective user and a drain section with an opening for the drainage of urine into a toilet. The drain member is folded in the longitudinal extent and connected together at the side margins and also at the holding part. The wall parts are adhered to one another or mechanically connected to one another. The wall parts can also be designed so that each has an arch extending transversely to the longitudinal extent of the drain member.

The known aids have an inlet section with wall parts which lie against each other or which are arched. In order to produce the connection the use of pressure is necessary which means that the wall parts not only lie against each other but also stick to one another. The aids, are removable in rolled or stacked form, are individually removable from a dispenser. The adhesion of the wall part is enhanced and the arching is reduced through the coil density and the packing density, in particular with longer storage.

It is regarded as disadvantageous that the inlet section has to be opened before use by manual spreading and that a larger waste arises during manufacture.

The invention is based on the object of providing an aid which is in particular improved in this respect and is simpler to handle.

This object is satisfied in accordance with the invention by the features of the claims.

The aid formed in accordance with the invention contains a drain member improved relative to the known embodiment which has a double-folded inlet section which can be automatically opened by pivoting of the holding part and which can be produced automatically and at favourable cost as a result of the low material waste.

Advantageous embodiments result from the dependent claims.

The invention will be explained in the following with reference to the accompanying drawings. There are shown:

FIG. 1 a side view of a first embodiment of an aid in accordance with the invention;

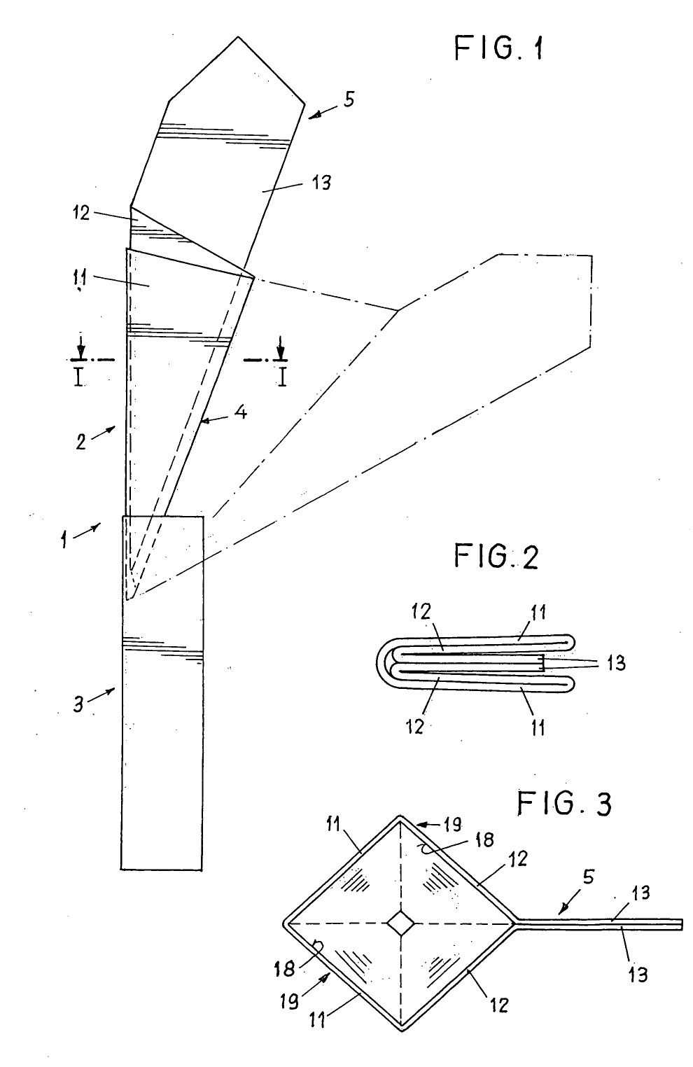

FIG. 2 a section along the line I-I in FIG. 1;

FIG. 3 a view of the aid of FIG. 1 in the state of use;

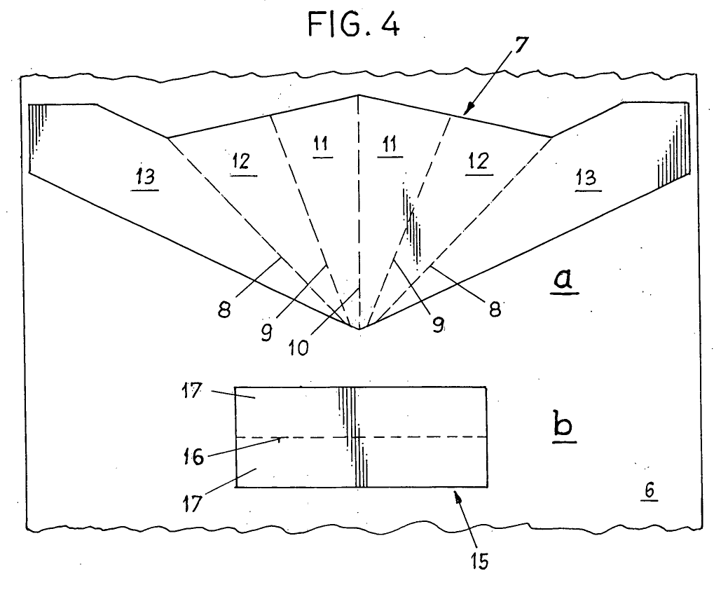

FIGS. 4a/b a cut blank of the embodiment of FIG. 1;

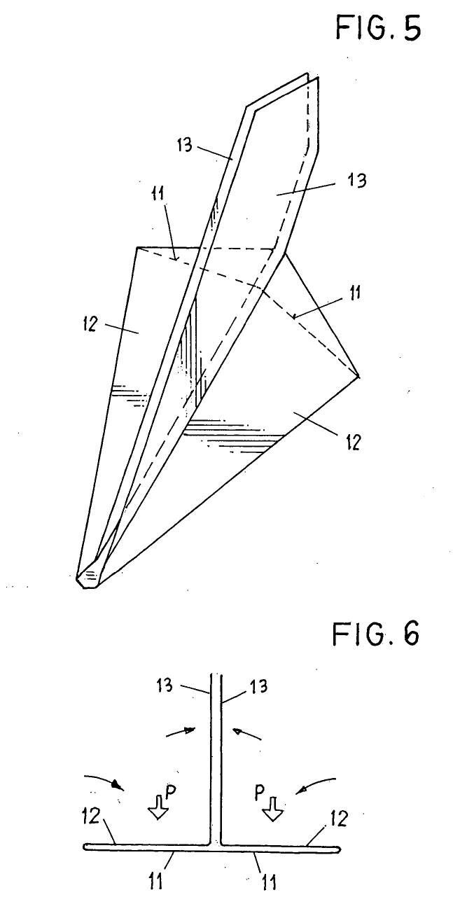

FIG. 5 an introduction section in accordance with FIG. 1 in accordance with a first method step in a perspective illustration;

FIG. 6 a cross-section through the folded introduction section of FIG. 5;

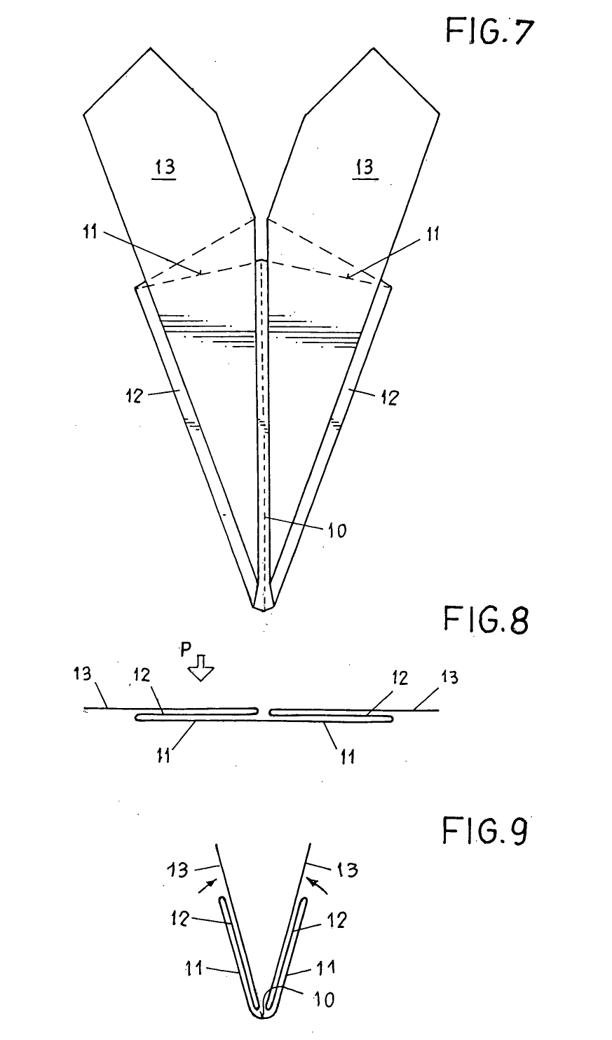

FIG. 7 a plan view of the introduction section in accordance with a second method step;

FIG. 8 a cross-section through the folded introduction section of FIG. 7;

FIG. 9 a cross-section of the introduction section in the third method step;

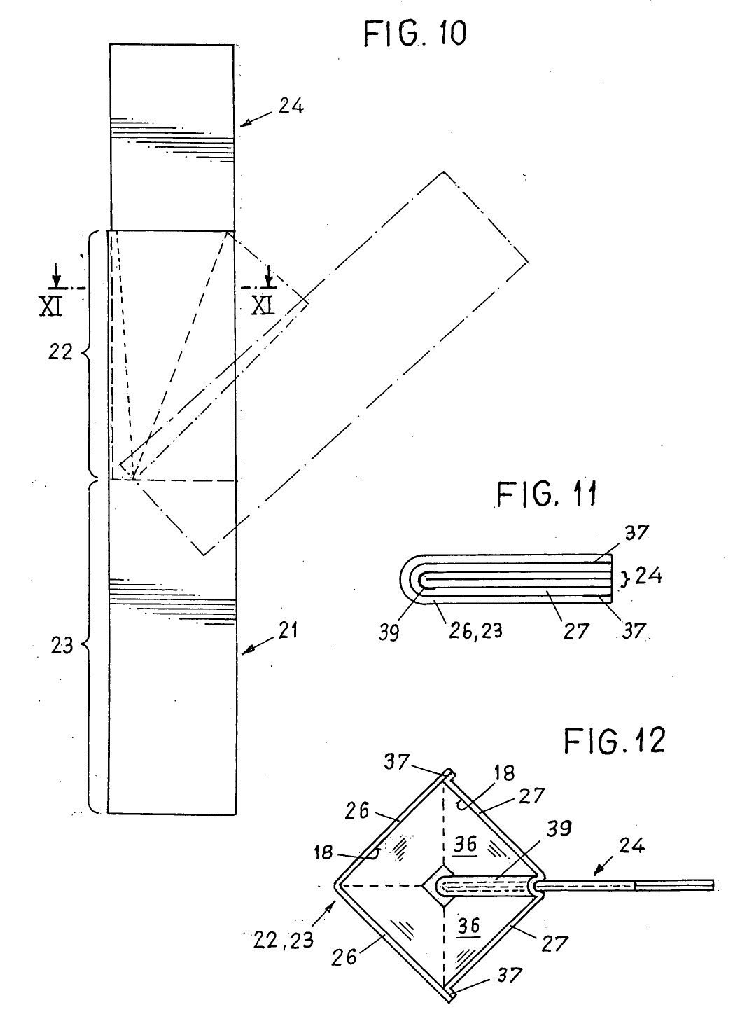

FIG. 10 a side view of a second embodiment of an aid in accordance with the invention;

FIG. 11 a section along the line XI-XI in FIG. 10;

FIG. 12 a view of the aid of FIG. 10 in the state of use;

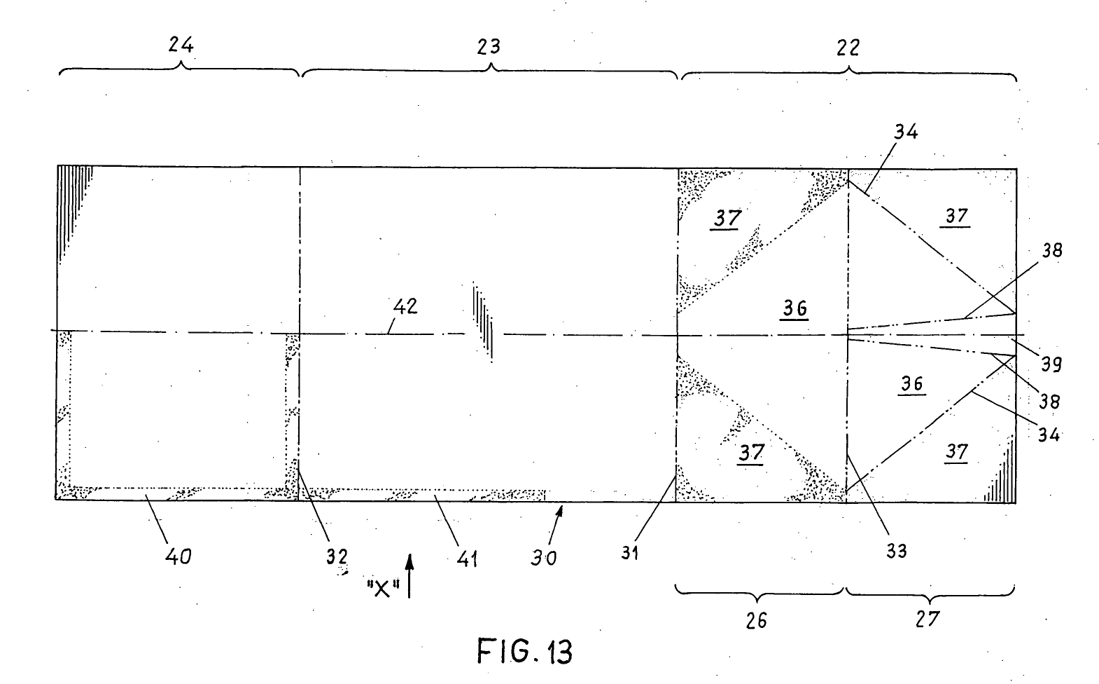

FIG. 13 a cut blank of the embodiment of FIG. 10;

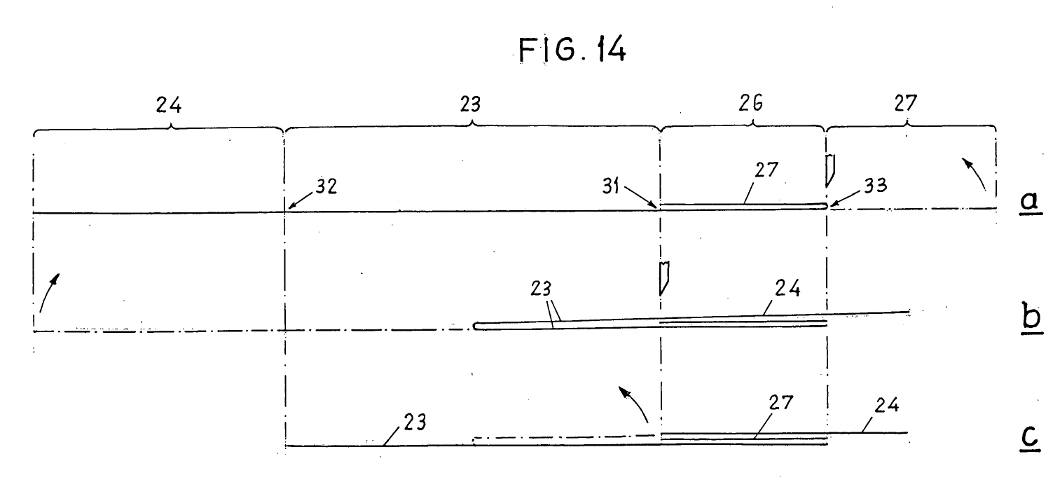

FIGS. 14a-c schematic representations of the method steps for manufacture of the second embodiment of FIG. 10;

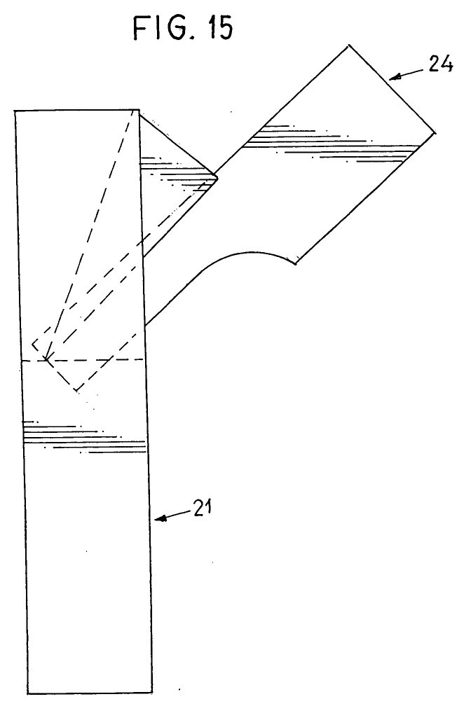

FIG. 15 a modified form of the aid of FIG. 10;

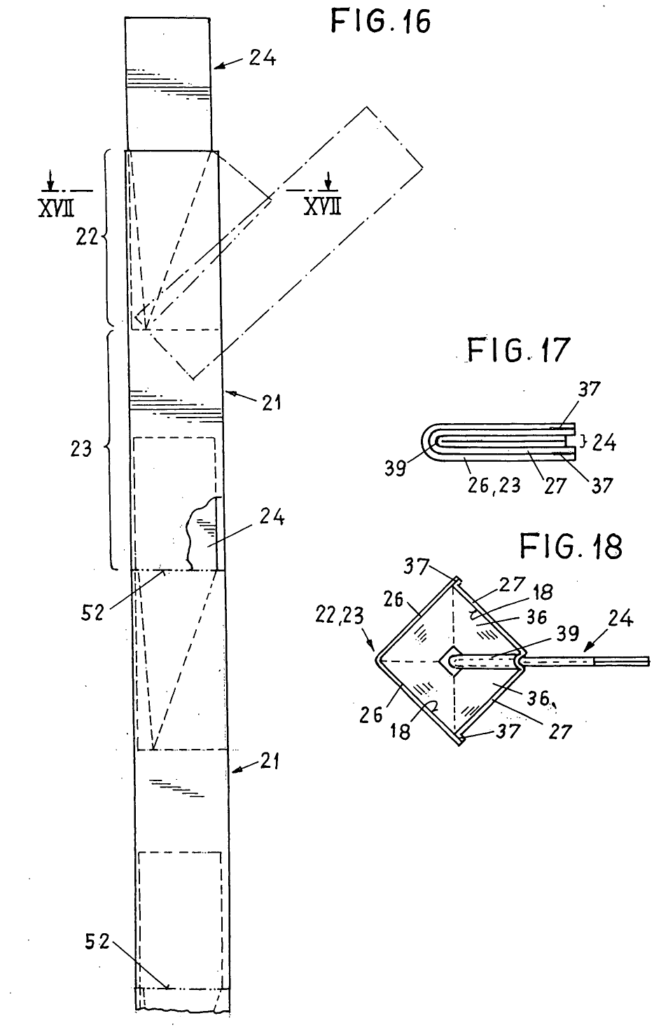

FIG. 16 a third embodiment of the aid of the invention;

FIG. 17 a section along the line XVII-XVII in FIG. 16;

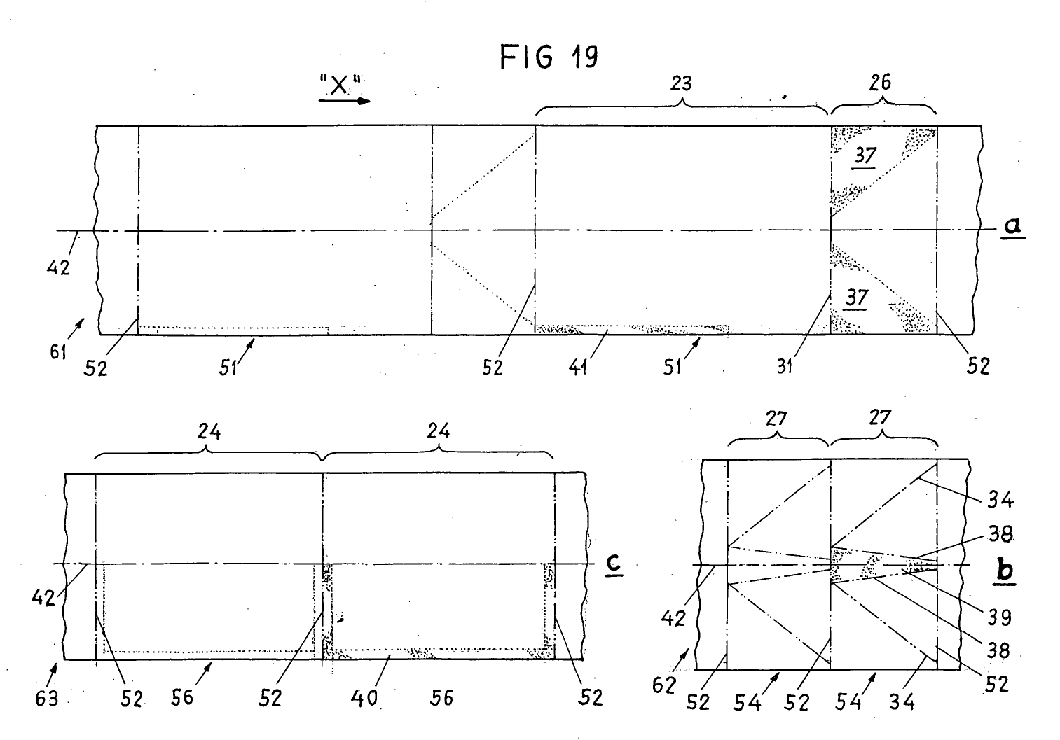

FIG. 18 a view of the aid of FIG. 16 in the state of use;

FIGS. 19a-c three cut blanks of the embodiments of FIG. 16;

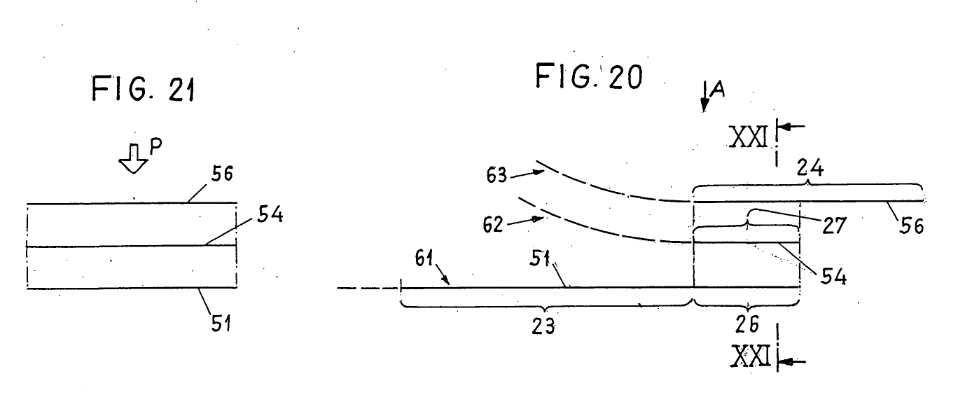

FIG. 20 a schematic representation of the method steps for the assembly of the embodiment of FIG. 16;

FIG. 21 a section along the line XXI-XXI in FIG. 20 and

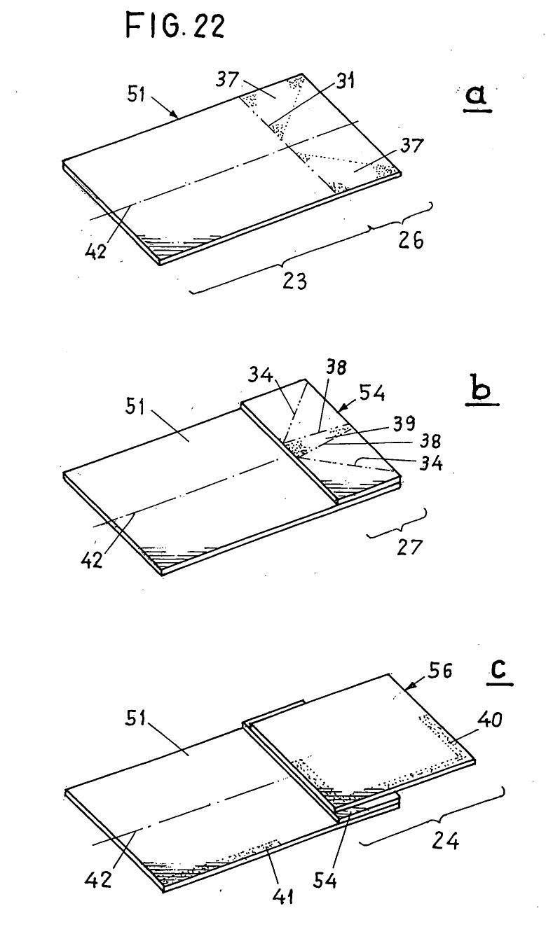

FIGS. 22a-c a perspective representations of the method steps 1 to 7 for the assembly of the embodiment of FIG. 16.

Reference is made to the FIGS. 1 to 9 which show a first embodiment of the aid. The aid consists of hygiene paper and is formed as a drain member 1. The drain member consists of an inlet section 2 and a drain section 3 which are connected together. The inlet section 2 has a doubly folded section 4 with a holding part 5 which projects beyond the folded section 4 and is surrounded by the folded section 4. The drain section 3 consists of a singly folded section. The inlet section 2 and the drain section 3 are formed from a paper web 6 with the cut blanks 7, 15, shown in FIGS. 4a and 4b. It is however also possible to form the cut blanks for the inlet section 2 and for the drain section 3 from two paper webs.

As shown in FIG. 4a, the cut blank 7 for the inlet section 2 is symmetrical and is subdivided by grooves 8, 9 and 10 into sections 11, 12 and 13, with in each case two sections 11, 12 and 13 being identically formed. The sections 11 and 12 are provided for the formation of the funnel and the sections 13 are provided for the formation of the holding part. As shown in FIG. 4b the cut blank 15 for the drain section 3 is subdivided by a groove 16 into two like sections 17.

As FIG. 2 shows the inlet section 2 is multiply folded along the grooves 8 and 9 provided in the cut blank so that the sections 13 are disposed towards the inside, the sections 12 contact the sections 13 and the sections 11 contact the sections 12. The sections 13 are connected to one another over the full area and the sections 11 and engage around the sections 12.

The manufacture of the above-described drain member takes place with a laying in process which is characterized by method steps independent of one another and indeed: manufacture of the cut blanks 7 and 15 for the inlet section 2 and for the drain section 3. Manufacture of the inlet section 2. Manufacture of the drain section 3 and connection of the inlet section 2 to the drain section 3.

In order to manufacture the cut blanks 7 and 15 a paper web 6 is continually supplied in the direction of the arrow X, is provided with the grooves 7, 8, 9 and 10 and the cut blanks are stamped out from the paper web 6. For the further processing, the cut blanks are separated.

For the manufacture of the inlet section 2 the cut blank 7 is processed with the following method steps. In a first step the sections 12 and 13 are simultaneously so folded along the grooves 8 and 9 that the sections 12 respectively lie on the adjacent sections 11 and the sections 13 each project perpendicularly and parallel to one another away from the sections 12 and thereafter the folds are fixed by means of pressure P (FIG. 5, 6). In a second step the sections 13 are folded apart so that the sections 13 contact the sections 12 (FIG. 8). FIG. 7 shows the freely exposed surfaces of the sections 13 onto which an adhesive is applied. In a third step the previously folded device is so folded along the groove 10 that the sections 13 contact one another (FIG. 9). Finally, the entire construction is fixed with pressure.

For the manufacture of the drain section the cut blank 15 is provided in a fourth method step with adhesive at an end section and is subsequently folded along the groove 16 so that the folded sections are spread apart. Finally the end section of the drain section carrying adhesive is pushed onto the inlet section 2 and subsequently connected with this to form the finished aid by means of pressure (FIG. 1).

Reference is made to FIGS. 10 to 15 which show a second embodiment of the aid. The aid consists of a hygiene paper and is formed as a drain member 21 which has an inlet section 22, a drain section 23 and a holding part 24. The inlet section 22 is formed of a first section 26 which is formed in one piece with the drain section 23 and of a second section 27 which is connected to the first section 26. The holding part 24 is connected to the second section 27 and projects beyond the inlet section 22. The inlet section 22, the drain section 23 and the holding part 24 are formed from the cut blank shown in FIG. 13.

As FIG. 13 shows the cut blank 30 for the drain member 21 is strip-like and is subdivided by grooves 31 and 32 into a section for the formation of the inlet section 22, a section for the formation of the drain section 23 and a section for the formation of the holding part 24. The section 22 is subdivided by a groove 33 into the sections 26 and 27. The section 27 is provided with two grooves 34 which respectively subdivide the section 27 into a section 36 and two sections 37. The section 27 is furthermore provided with two grooves 38 which bound a section 39 for the attachment of the holding part 24.

The manufacture of the above-described drain member takes place in the direction of the arrow X with a cycle time free and abutment free method which includes the following method steps.

In a first step a paper web 6 is continually supplied, is provided with grooves 31, 32, 33, 34 and 38 and the cut blank 30 is stamped out. In a second step adhesive is applied (FIG. 13) to the sections 37 of the section 26. In a third step the section 27 is folded along the groove 33 so that the sections 26 and 27 lie against one another and thereafter the fold section between the sections 26 and 27 is cut off so that an opening arises (FIG. 14a). In a fourth step the sections are connected to one another by means of pressure and an adhesive is applied onto the folded section 27 in the section 39. In a fifth step the section 23 is turned over so that the groove 32 covers the groove 31, the section 24 is cut off and the section 23 is turned back again (FIG. 14b). In a sixth step the now freely exposed rear side of the section 24 is provided with an adhesive in the section 40 and the section 23 is provided with an adhesive in section 41 and the section 24 is also simultaneously connected to the section 27 (14c). In a seventh step the folded cut blank is folded along the middle line 42 so that the sections 23 and 24 contact one another and are connected together by means of pressure. Instead of the paper web 6 a paper strip can also be supplied.

FIG. 15 shows an aid 21 with a modified holding part 21.

The aids are interleaved into one another and packed in a dispenser. The dispenser has an opening for the removal of an aid. The opening is so designed that the holding part 5 or 24 projects out of the dispenser and the edges formed by folding of the sections 26 and 27 contact the wall parts of the dispenser which bound the opening of the dispenser. In this way the aid can be removed from the dispenser by means of the holding part 5 or 24 with simultaneous opening of the funnel-like introductory section. From this the advantage arises that the aid is present in the state ready for use.

A modification of the second embodiment of the aid will be described with reference to the FIGS. 16 to 22. The aid consists of hygiene paper and is formed as a drain member 21. The drain member has the same features as the embodiment in accordance with the FIGS. 10 to 12. In the following only the differing features will be described. As FIG. 16 shows a plurality of drain members 21 are formed following one another, with the holding part 24 of the following drain member 21 lying within the drain section 23 of the preceding drain member 21. For this purpose the holding part 24 must have a small width such as is evident from the FIG. 17. The drain members 21 are so connected to one another via a perforation 52 that the aids can be separated by tearing them off individually. The inlet section 22, the drain section 23 and the holding part 24 are formed form the three sections 51, 54, 56 shown in the FIGS. 19a to 19c which are respectively formed following one another on one strip.

As the FIGS. 19a to 19c show a first cut blank 51 is bounded by two perforations 52 for the separating of the drain members 21 and is subdivided by a groove 31 into the section 26 for the forming of a part of the inlet section 22 and the drain section 23 (FIG. 19a). A second cut blank 54 forms the section 27 for the formation of the other part of the inlet section 22 and is bounded by two perforations 52 (FIG. 19b). A third cut blank 56 for the formation of the holding part 24 is bounded by the perforations 52.

The manufacture of the above described drain member likewise takes place in the direction of the arrow X with a method which is cycle time free and abutment free. As shown in the FIGS. 20, 21 three paper strips 61. 62 and 63 are supplied staggered and the drain member 21 is manufactured with the following method steps.

In a first step the first paper strip 61 is continuously supplied, is perforated along the line 52, is provided with the groove 31 and perforated along the line 52. In a second step an adhesive is applied to the sections 37 of the section 26. Thus the first cut blank 51 is prepared (FIG. 19a). In a third step the second paper strip 62 is perforated along the line 52, is provided with the grooves 34 and 38 and perforated along the line 52. Thus the second cut blank 54 is prepared. In a fourth step the section 27 is laid onto the section 26, is separated from the paper strip 62 and the sections 26, 27 are connected by means of pressure (FIG. 20). In a fifth step a third paper strip 63 with a smaller width is perforated along the lines 52 and is provided with an adhesive in the region 40. Thus the third cut blank 56 is prepared. In a sixth step the section 24 is separated from the third paper strip 63 and is laid onto the section 27 so that the cut edge is flush with the cut edge of the section 27 and the holding part 24 projects beyond the second cut blank and projects beyond a section of the previously formed drain member (FIG. 20). In a seventh step the section 27 is provided in the section 39 with an adhesive and the section 23 is provided in the region 41 with an adhesive. In an eighth step the whole is folded along the centreline 42, the sections 23 and 24 are laid apart from one another and connected together.

As FIG. 22 shows the regions 40 and 41 provided with an adhesive are exposed after assembly and are placed on one another after folding along the line 42 and connected to one another by means of pressure.

The attached aids are arranged in a dispenser, either in roll form or continuously folded in zigzag, so that the aids can be removed individually.

Instead of the adhesive 37 the regions can also be connected by a mechanically produced connection, for example by inter-engaging embossed features.

The layer 18 can be formed by an impregnation with an environmentally friendly, decomposable, material which rapidly dissolves and/or decomposes on contact with water, for example with a soap, a grease or the like. The layer 18 can furthermore include a mechanical test agent, for example an indicator which reacts to the acidic or basic nature of the urine of the respective user. Drain elements 1 and 21 executed in such a way can thus also be used as a simple means for corresponding medical checking functions which are, for example, required daily.

The drain members 1 and 21 serve to facilitate urination in a standing position of the respective user and for the draining of urine into a non-illustrated toilet. The aid is also in particular suitable for use in private or other toilet systems, for example those of transport means in which no urinal is present.

The disposable aid is formed as a folded drain member 21 with walls of a hygiene paper which is formed by folding along a central line. The drain member 21 contains an inlet section 22 for the reception of urine, a drain section 23 for the draining of urine into a toilet or the like and a sideways projecting holding part 24 which is connected to the inlet section 22. The inlet section 22 is formed of two wall sections of the same design 26; 27 and can be formed by them into a funnel. A plurality of drain members 21 can be manufactured individually or in roll form and packed into a dispenser. The drain members can be made at favourable cost and enable a comfortable, hygienically advantageous use as well as a simple disposal.

1. A disposable aid for urinating having a folded drain member (1) which is formed from walls (19) which lie flat against each other, of a material which can soften and/or decompose in water, by folding in the longitudinal direction, said drain member (1) including and inlet section (2) which can be formed into a funnel consisting of two wall sections (19) provided with a wetting inhibiting layer (18) for reception of urine, a drain section (3) for the draining of urine into a toilet or the like and of two wall sections (17) free of wetting inhibiting medium and a sideways projecting holding part (5) formed of two wall sections (13), the holding part being formed in one piece with the entry section (2), characterized in that the walls (19) forming the inlet section (2) have two wall sections (11, 12) which are folded along lines (8; 9) such that the wall sections (11; 12) are arranged lying over one another and in that the holding part (5) for the opening of the inlet section (2) is surrounded by the wall sections.

2. Aid in accordance with claim 1, characterized in that the inlet section (2) is formed of a first cut blank (7) and the drain section (3) of a second cut blank (15) and in that the inlet section (2) and the drain section (3) are connected to one another.

3. Aid in accordance with claim 1, characterized in that the first cut blank (7) includes the inlet section (2) and the holding part (4) and in that the wall sections (13) forming the holding part (4) are connected to one another over their full area.

4. Disposable aid for urination having a folded drain member (21) which is formed of walls which lie flat against each other, of a material which can soften and/or can decompose in water, by folding along the central line (42) and which bound a drain channel provided with a wetting inhibiting layer (18), said drain member (21) including an inlet section (22) which can be formed into a funnel for the reception of urine, a drain section (23) for the draining of urine into a toilet and a sideways projecting holding part (24), characterized in that the inlet section (22) is formed of a first wall with a wall section (26) which is made in one piece with the wall section (23) for the formation of the drain section (23) and of a second wall with a wall section (27) and in that the holding part (24) is connected to the wall section (27) for the forming of the inlet section (21) and is surrounded by the wall sections.

5. Aid in accordance with claim 4, characterized in that the wall sections of the drain section (23) are at least partly connected to one another in the edge region.

6. Aid in accordance with claim 4, characterized in that the drain member (21) is formed of a third cut blank (30).

7. Aid in accordance with claim 4, characterized in that the drain member (21) is formed of a fourth, fifth, and sixth cut blank (51, 54, 56).

8. Aid in accordance with claim 4, characterized in that the wall section (11, 12, 26, 27) are provided with stiffening grooves.

9. Aid in accordance with claim 4, characterized in that the drain member consists of a hygiene paper.

10. Aid in accordance with claim 4, the wetting inhibiting layer (18) contains a medical test agent which reacts to a specific characteristic of the urine of the respective user.

11. Aid in accordance with claim 1, characterized in that the wall section (11, 12, 26, 27) are provided with stiffening grooves.

12. Aid in accordance with claim 1, characterized in that the drain member consists of a hygiene paper.

13. Aid in accordance with claim 1, the wetting inhibiting layer (18) contains a medical test agent which reacts to a specific characteristic of the urine of the respective user.
